(19) United States
(12) Patent Application Publication
(10) Pub. No.: US 2007/0155812 A1
(43) Pub. Date: Jul. 5, 2007

(54) Thio-substituted phenbutazone compounds as anti-inflammatory, anti-viral and immunomodulatory agents

(76) Inventor: David W. C. Dekkers, Pastoor Verhoevenstraat 6 (NL)

Correspondence Address: BACON & THOMAS, PLLC, 625 SLATERS LANE, FOURTH FLOOR, ALEXANDRIA, VA, 22314, US

(21) Appl. No.: 10/565,507

(22) PCT Filed: Jul. 23, 2004
(86) PCT No.: PCT/GB04/03210

§ 371 Date: Oct. 2, 2006

(30) Foreign Application Priority Data
Jul. 23, 2003 (GB) 0317269.9

(51) Int. Cl. A61K 31/4152; C07D 231/32

(52) U.S. Cl. 514/404; 548/366.4

(57) ABSTRACT

The present invention relates to optionally substituted compounds of the formula I or a salts thereof; 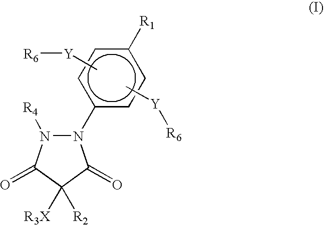
wherein R₁ is O or S, R₂ is hydrogen or a C₁-C₁₀ organic group attached by a carbon atom, X is H, O, OO, S or SS R₃ is absent where X=H, is hydrogen or is a hydroxyl or thiol protecting group, R₄ is a hetero- or homo-cyclic aryl group, optionally substituted with a further group R₅, one Y group is S and the other is either H (in which case only one R₆ group is present) or S; and R₆ is, independently, an organic group of molecular weight up to around 500 amu. The invention also provides compositions comprising such compounds and methods of treatment comprising administration of such compositions.

The present invention relates to certain substituted 4-hydroxyoxyphenbutazone compounds and the use thereof in therapy. More particularly, the present invention relates to such compounds and their use as anti-inflammatory, anti-viral and immunomodulatory agents.

The cyclic pyrazolidine dione compounds phenbutazone, oxyphenbutazone and 4-hydroxy oxyphenbutazone are known or suggested as having anti-inflammatory, antiviral and/or immunomodulatory properties.

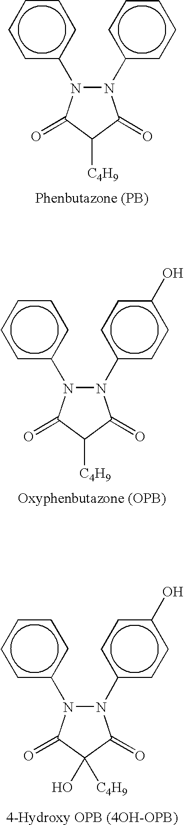

Many derivatives of these pyrazole based structures have been investigated, including derivatives (e.g. U.S. Pat. No. 3,968,219), and prodrugs (e.g. U.S. Pat. No. 4,117,232, U.S. Pat. No. 3,957,803, U.S. Pat. No. 4,169,147, U.S. Pat. No. 4,036,845 and U.S. Pat. No. 4,139,709). The principal work on those with biological activity has, however, related to varying the makeup of and substituents on the central pyrazolidine core.

The present inventors have now, unexpectedly, established that a new class of related compounds exists in which at least one aromatic ring is substituted with at least one thiol.

In a first aspect, the present invention therefore provides an optionally substituted compound of the formula I, or a salt thereof. Particular examples include compounds (Ia) and (Ib) 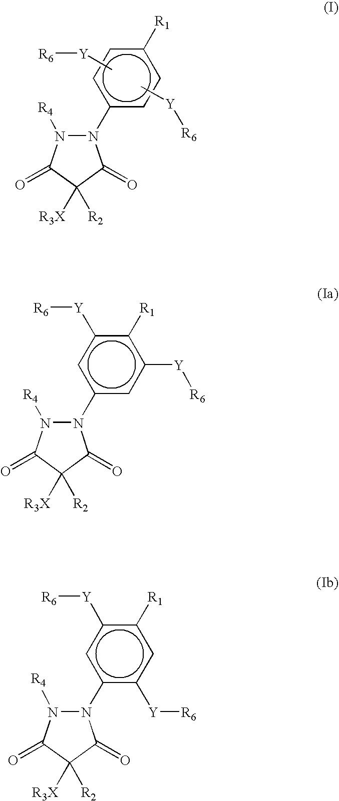
wherein R₁ is OH, SH, O-alkyl, S-alkyl, O-acyl or S-acyl; R₂ is hydrogen or more preferably an C₁-C₁₀ organic group attached by a carbon atom, e.g. an optionally substituted alkyl, alkenyl, alkynyl, alkaryl, aralkyl, arylsulphonylalkyl or aralkenyl group; X is H, O, OO, S or SS; R₃ is absent (where X=H), is hydrogen or is a hydroxyl or thiol protecting group (e.g. a, preferably C₂-C₇, acyl, or alkaryl group, such as an acetyl or benzyl group), R₄ is a hetero- or preferably homo-cyclic aryl group, optionally substituted with a further group R₅ (e.g. with an alkyl, alkenyl or alkynyl group, OH, O-alkyl, thio, thioalkyl, halo, or primary, secondary, tertiary or quaternary amino group); one Y group is S and the other is either H (in which case only one R₆ group is present) or S; and R₆ is (independently, where 2 R₆ groups are present) an organic group of molecular weight up to around 500 amu, such as a substituted or unsubstituted alkyl, alkenyl, alkynyl, alkaryl, aralkyl, alkyl ester, alkyl amide, alkyl acid, polyol, sugar, oligo(alkylamide), oligo(alkylester), or oligopeptide group. Where more than one R₆ group is present, these may be the same or different.

The present inventors have also established that compounds of the invention may be conveniently prepared by the reaction between thiols and certain well known starting materials.

In a further aspect, the present invention therefore provides a method for the synthesis of a compound of the invention by reaction of a thiol (R₆—SH) with a starting material of formula II

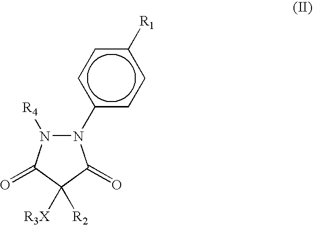

Wherein R₁, R₂, R₃, R₄ and X are as herein described, or protected derivatives or precursors thereof. Such starting materials are typically oxyphenbutazones or 4-hydroxy-oxyphenbutazones and are synthesised by methods described herein and by methods known in the art, such as from WO 01/00585 and the references cited therein. The disclosure contained in this document and in all references cited herein is hereby incorporated herein by reference.

Specifically, 4-hydroxyoxyphenbutazones may be synthesised from oxyphenbutazones by oxidation of corresponding compounds in which the R₃X position is occupied by hydrogen; from other 4-OH OPBs by reaction of corresponding compounds in which R₃X is HX with hydroxy or thiol protecting groups to introduce non-hydrogen R₃ group, or by condensation of a hydrazine compound with )an optionally protected 2-hydroxy-propane dioic acid halide, ester or similar compound, e.g. 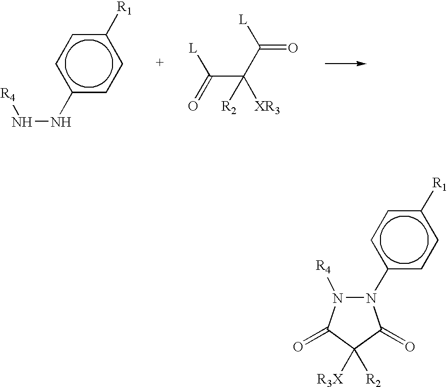
wherein R₁-R4 and X are as defined above for the starting materials and the groups L are leaving groups such as halides etc. Where X is H, oxyphenbutazones will result, which may be converted to 4-OH OPBs as described above. Where X is 0, 4-OH OPBs will be formed directly.

As will be readily appreciated, the hydrazines may be prepared by hydrogenation of the corresponding diarylazo compounds (since R₄ is aryl), which in turn can be synthesised from simple aromatic nitro compounds in the presence of LiAlH₄

Evidently, the compounds of the present invention may also be made directly by reaction with appropriate hydrozine compounds already having the Y-R₆ substitution, or a protected equivalent attached to the aromatic ring thereof;

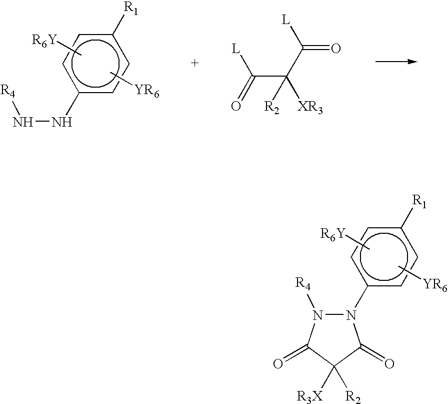

The thiol molecule may be represented by R₆-SH, where R₆ is as defined above, a protected equivalent or a precursor thereof. Preferred R₆ groups for reacting to form the molecules of the invention will be the preferred R₆ groups indicated herein.

In a further aspect, the present invention provides a compound obtained or obtainable by reaction of a thiol (R₆-SH as defined herein) with starting material of formula II as defined herein. Preferably, such compounds are obtainable by reaction of a preferred starting material with a preferred thiol as defined herein.

The present inventors have further, unexpectedly, established that compounds of the present invention have considerable utility as modulators of inflammatory and immune reactions within the body and in the treatment of certain conditions, particularly viral, neoplastic, inflammatory, allergic and autoimmune conditions. The compounds of the present invention may also provide a “tonic” effect in subjects suffering from fatigue, lethargy or the effects of aging, whether or not any direct, identifiable, cause of these symptoms is evident.

In a further aspect, the present invention therefore provides a method of treatment of a mammalian (preferably human) subject comprising administering a compound of formula I or a salt thereof as defined herein. In a preferred embodiment, the present invention provides a method of treatment of a viral, neoplastic, inflammatory, allergic or autoimmune condition (particularly disease) or of the symptoms of fatigue, lethargy or old age by administration of at least one compound of the present invention. Preferably, the compound will be a preferred compound, as described herein.

In a further aspect the present invention provides a compound of formula I or a salt thereof as defined herein for use in therapy. Preferably, the compound will be a preferred compound as described herein.

In a still further aspect, the present invention provides the use of a compound of formula I or a salt thereof as defined herein in the manufacture of a medicament. Preferably, this will be a medicament for the treatment of a viral, neoplastic, inflammatory, allergic or autoimmune condition (particularly disease) or of the symptoms of fatigue, lethargy or old age. Preferably, the compound will be a preferred compound as described herein.

Compounds of the present invention may be usefully administered in the form of a pharmaceutical composition, particularly for the treatment of disease. Alternatively, the compounds of the present invention may be taken in the form of an “functional food”, a supplement or as a food or beverage fortification, particularly where a “tonic” effect in the reduction of the symptoms of fatigue, lethargy or old age or a general boost to the immune system is desirable.

In a yet still further aspect, the present invention therefore provides a pharmaceutical composition comprising a compound of formula I or a salt thereof as defined herein and at least one pharmaceutically acceptable excipient, carrier or diluent. The invention also provides a functional or fortified food comprising a compound of formula I or a salt thereof formulated in an edible food or beverage.

The compounds of the present invention may furthermore be useful in the synthesis of certain open-chain derivatives thereof, which may also show any of the therapeutic effects described herein. Such compounds may have formula VI; 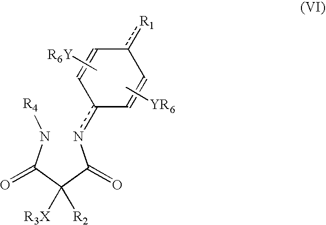
and are formable by the reaction of compounds of the present invention, especially by hydrolysis under mildly basic conditions. The use of the compounds of the present invention in the (in vitro or in vivo) synthesis of, or as precursors of, such open-chain derivatives thus forms a further aspect of the present invention, as does a method of synthesis of such open-chain compounds (in vitro or in vivo) from those of the invention. Preferred compounds of the invention as described herein will form preferred open-chain compounds and are thus preferred in this method.

Preferred compounds of the invention are of formula III, and salts thereof, particular examples include compounds IIIa and IIIb; 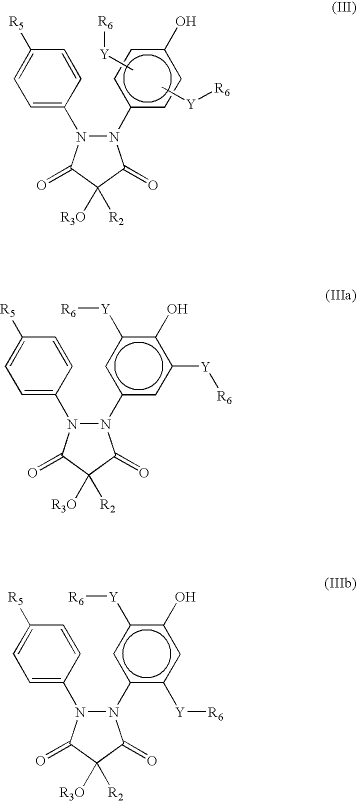
wherein Y, R₂, R₃ and R₆ are as described above and R₅ is is an alkyl, alkenyl or alkynyl group (such as those listed infra far R₂), an OH, O-alkyl, O-acyl, SH, S-alkyl, S-acyl, halo, or primary, secondary, tertiary or quaternary amino group. Preferred R₅ groups are hydrogen, OH and O-acyl (e.g O-acetyl). Most preferred are hydrogen, OH and O-acetyl.

In the compounds and starting materials of the invention, R₂ is preferably a C₁ to C₆ alkyl, alkenyl, or alkynyl group (e.g. a methyl, ethyl, ethylenyl, acetylenyl, n-propyl, i-propyl, prop-l-enyl, prop-2-enyl, n-butyl, i-butyl, s-butyl, t-butyl, but-1-enyl, but-2-enyl, but-3-enyl, 1-methyl-prop-l-enyl, 1-methyl-prop-2-enyl, 2-methyl-prop-1-enyl, 2-methyl-prop-2-enyl, n-pentyl, i-pentyl etc.) or an arylsulphonylalkyl group such as phenylsuphonylmethyl. More preferably R₂ is C₂ to C₆ alkyl, particularly n-butyl, i-butyl, s-butyl or t-butyl. The most preferred R₂ group is n-butyl.

R₃ in the compounds and starting materials described herein is preferably hydrogen or a metabolically labile protecting group which yields a physiologically tolerable byproduct. Suitable protecting groups are acyl groups, particularly acetyl, propanoyl, methylpropanoyl or n-butanoyl. Many additional OH and SH protecting groups are however known (see e.g. Greene, “protective groups in organic synthesis”, Wiley Interscience, NY, 1981) and these may be of value as products, or particularly as intermediates. Most preferred R₃ groups are hydrogen and acetyl.

In the compounds present invention, one Y-R₆ group may be hydrogen but at least one (and preferably both) is a thiol group substituted with an R₆ group, where R6 is a targeting moiety (including, for example an antibody, antibody fragment, receptor or receptor fragment) or a small (esp MW<500) organic group having at least two functional groups selected from esters, amides, carboxylic acids, hydroxyl groups and amines. Where two R₆ groups are present, these may be the same or different. Preferably, R₆ is an oligo ester or oligo peptide (i.e. a moiety having at least one peptide bond) with at least one free acid and/or amine group. Examples of such groups include specific binding peptides such as antibody fragments. More preferably, at least one Y-R₆ groupis a 1-5 residue oligo peptide. Most preferably, at least one Y-R₆ group is glutathione or cysteine. That is to say, for example, both Y-R₆ groups may be glutathione, or one may be glutathione and the other hydrogen.

The most preferred compound of the present invention is of formula IV or a salt thereof; 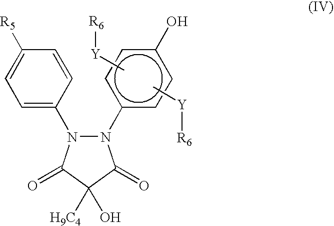
wherein R₅ is hydrogen or OH and at least one R₆-Y group is a glutathione moiety attached via the sulphur atom thereof. Preferred compounds of formulae I, III and IV include a) those with both -Y-R₆ groups ortho to R₁ and b) those with one -Y-R₆ group ortho to R₁ and one -Y-R₆ group meta to R₁.

Preferred methods for the synthesis of compounds of formula I comprise condensation of thiols (R₆-SH) with starting materials of formula (V); 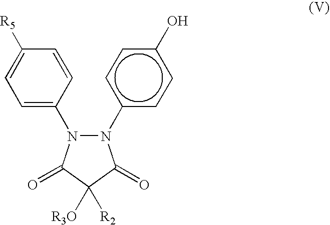
wherein the groups R₆, R₂, R₃ and R₅ are as described above and are preferably the preferred groups described above. In the most preferred starting material, R₁ is OH, R₂ is C₄H₉ (preferably n-butyl), R₃ is H and R₅ is H or OH. Preferred thiols are those of formula R₆-SH where R₆ is a preferred R₆ group as defined herein.

The condenation of the starting materials with a thiol may be carried out in aqueous solution, particularly in neutral or slightly basic aqueous solution at temperatures between 0° C. and 100° C., preferably between 20° C. and 60° C. for a period of 30 seconds to 4 hours, preferably 4 minutes to 1 hour, most preferably 10 to 45 minutes. In some cases the compound of formula I resulting from the ring-opening reaction will be labile to hydrolysis but will generally have a longer lifetime than its rate of formation from the starting material. In such cases, the reaction time will preferably be shorter than the half-life of the product under the conditions of the reaction.

The progress of the ring-opening reaction will be conveniently followed by techniques well known in the field of organic chemistry such as Nuclear Magnetic Resonance (NMR) spectroscopy, Infra-Red (IR) spectroscopy and/or mass spectrometry.

Medical conditions suitable for treatment, prevention or control by administration of the compounds, formulations, compositions or medicaments of the present invention include viral, neoplastic, autoimmune, inflammatory and allergic conditions including those which are secondary to other conditions and those having a viral, neoplastic, autoimmune, inflammatory or allergic component. Examples of immune, autoimmune, inflammatory and allergic conditions or conditions having a contribution from these mechanisms include Addison's disease, allergic conditions such as hay fever, food (e.g. nut, wheat or seafood) allergies or skin alergies, Alzheimer's disease, amyloidosis, (such as that resulting from conditions such as arthritis or tuberculosis), ankylosing spondylitis, asthma, antiplastic anemia, Behget's disease, Bechterew's disease, Cogan's syndrome, Crohn's disease, dermatomyositis, diabetes mellitus, eczema, glandular disorders (such as diabetis, especially type II, and hypo- or hyper-thyroidism), glomerulonephritis, haemolytic anemia, Hepatitis Huntinton's disease, inflammatory bowel diseases such as irritable bowel syndrome, immune suppression (such as due to infection with HIV, compromised bone marrow function, treatment with cytotoxic chemotherapeutic agents etc.), liver diseases such as autoimmune hepatitis or primary biliary cirrhosis, lung diseases such as interstitial lung disease, lupus erythematosus, Morbus Reiter, neoplastic disease (such as benign or particulatrly malignant neoplasms e.g cancer (sarcoma or carcinoma), leukemia etc.), neurological disorders such as multiple sclerosis or myasthenia gravis, inflammatory or autoimmune ocular disorders such as scleritis or uveitis, post-operative ocular inflammation, or resulting from Behcet's disease, osteoarthritis, Parkinson's disease, pemphigus, polyglandular deficiency, polymyositis, pernicious anemia, psoriasis, rheumatoid arthritis and other rheumatic disorders (such as Besnier's rheumatism, rheumatic fever, lumbago, or Poncet's rheumatism), sarcoidosis, scleroderma, Sjögren's syndrome, testicular failure, thrombocytopenic purpura, tissue rejection and prevention thereof, ulcerative colitis and Wegner's granulomatosis. Examples of viruses and viral conditions whiph may be treated, prevented and controlled include viral infections of CD4 cells (e.g. HIV-1, HIV-2, HTLV-I, HTLV-II and herpes viruses), togaviridea, reoviradea, picornaviradea, hantaviridea, orthomyxoviridea, paramyxoviridea, mononegaviralis, viral hepatitis, haemorrhagic fever, flaviviridea, viral encephalitis, coronoviridea, calciviridea, adenoviridea, papoviridea, arboviridea, pox virus, rhabdoviridea, and arenaviridea.

The thiol derivatised compounds of the present invention are advantageous over known compounds because the positions containing thiol substituents are not availble for reaction with thiols in vivo. Consumption of thiols such as glutathione can damage or kill cells and thus, the compounds may show lower toxicity and/or fewer side effects than known compounds.

Where the compounds, compositions or medicaments of the invention are administered to combat primary or secondary diseases, these may be in combination with other active agents, either as a combined formulation or as separate formulations administered simultaneously or sequentially. In particular, where the compositions of the invention are administered to combat a secondary disease, this will typically be simultaneously with, or following, treatment for the primary condition. For example, the compositions of the present invention may be administered in combination with antiviral agents (such as nucleoside analogues) in order to combat the viral disease and provide improved quality of life for the subject.

Compounds of the present invention may be formulated as pharmaceuticals by methods well known in the art. These formulations will typically be oral formulation such as tablets, coated tablets (such as controlled release tablets), capsules, suspensions, solutions, syrups, powders, or emulsions but may be formulations for inhalation (such as powders or aerosols), transdermal absorption (such as patches) or for parenteral (e.g subcutaneous, intramuscular or intravenous) ocular or rectal administration in the form of, for example, sterile saline solutions, drops or suppositories. Where the treatment is to be, for example, to reduce the inflammatory reactions relating to asthma, inhalable preparations will be most suitable and for some allergic conditions such a hay fever, nasal sprays may be most effective. Equally, topical preparations such as drops, creams or gels will be more suitable for ocular conditions or localised skin conditions.

The compounds of formula I and salts thereof may be formulated with conventional pharmaceutical carriers, dilluents and/or excipients such as aqueous carriers (e.g. water for injections), binders, fillers, stabilizers, osmolality ajusting agents, effervescing agents, pH buffers and modifiers, viscositiy modifiers, sweetners, lubricants, emulsifiers, flavours, coating agents (e.g. gastric jusce resistant coatings) etc.

The dosage of the compounds of formula I or salts thereof administered to a subject will be dependent upon the species, size, maturity, health and condition of the subject, upon the severity of the condition and upon the formulation chosen. Inhalable or intravenous formulations, for example, may deliver a larger proportion of the active agent to the subject than oral formulations and topical treatment will typically require lower doses than systemic treatment. Generally, doses will be in the range of 0.05 to 2000 mg/day, more typically 0.2 to 1000 mg/day, especially 0.5 to 200 mg/day. Administration will typically be once, twice, three or four times per day but may more or less often (e.g. five or pix times per day, once every two or three days, or every time symptoms are detected) if appropriate. Topical treatment will typically be administered more often than systemic treatments.

Where the compounds of the present invention are administered as a tonic, such as to reduce lethargy, the symptoms of old age or to boost the immune system, they may be formulated as pharmaceuticals as above. Altermatively, the compounds may be formulated as functional foods or beverages, in which situation the carriers and excipients will typically be edible food or beverage products. Such products may be processed foods for consumption hot, such as ready meals but will more preferably be cold foods include spreads, jams, still or carbonated soft drinks, breads, biscuits, icecreams, chilled desserts such as yoghurts, mousses or trifles, milk or milk based drinks.

Where the comounds of the invention are formulated as functional foods or beverages, it will be important that the maximum dose which can be accidentally consumed by over-eating such foods is not excessive. In such cases, the dosage present in one portion of such functional foods will typically be no more than 5 000 times less than the lethal dose, more preferably no more than 10 000 times less and most preferably no more than 100 000 times less thant the human lethal dose.

Where the compounds of the invention are referred to herein as salts, these will generally be pharmaceutically acceptable salts i.e. those with physiologically tollerable counterions. Such ions include sodium, calcium, organic amines, halides (especially chloride), phosphates, hydrogen carbonates etc.

Without being bound by theory, the effect of the compounds, compositions and medicaments of the invention is believed in part to be the result of a stimulating and modulating effect upon certain aspects of the mammalian immune and defence systems, particularly, for example, by enhancing macrophage activity and white blood cell (WBC) activity and count and modulating levels of the acute phase proteins (APPs) such as C-reactive protein (CRP). The “tonic” effect of the compositions may therefore be, at least partially, attributable to a “cleanup” effect, in which the body is stimulated to remove not only infectious agents but also cell debris and other unwanted matter. In addition, and in spite of their effect as WBC stimulants, the compounds of the invention show effects as inhibitors of the production of certian cytokines and of T-lymphocyte and monocyte activation and modulators of interlukins. By such a processes, the tendency for the immune system to generate unwanted inflammation both in general and as a result of encountering biological debris is reduced, as is the danger of autoimmune reaction. As a result, the subject is provided with a better quality of life and the immune system is stimulated and the body purged of some unnecessary and even detrimental antigens. This tonic effect may be applied during or following treatment for a primary disease, condition or infection, or may be an end in itself, when, for example, infection, drug treatment or the aging process has resulted in compromised immune function or a build up of unwanted, immunogenic and/or inflammatory matter in the system.

The stimulation of systems such as certain APPs is believed to induce a cleanup of the system, removing cell debris that would otherwise stimulate inflammation and may present native antigens that could induce autoimmune responses. The breakdown products of host cells can also,induce the death of neighbouring cells, thereby causing a cascade of cell death and inflammation. Unusually, the compounds of the present invention typically stimulate acute phase proteins without inducing significant fever and are not typically general immune-suppressants.

Additional methods for bringing about a cleanup of biological debris include binding by certain plasma proteins such as particular immunoglobulins of type M (IgMs) with specificity for the membrane phospholipids of dead (but not living) cells, b2 glicoprotein I, clusterin and serum amyloid P. The activity of these mechanisms may also be modulated by the compounds of the present invention.

Diseases of collagen, such as systemic lupus, are for example believed to have a build up of cell debris as a primary cause in many cases. As a result, the compounds of the present invention are highly suitable for the treatment or prevention of collagenous disease, for example in those showing symptoms of the disease or those having a predisposition due to inheritance or injury.

Similarly, a build up of biological debris is a particular problem in Multiple Sclerosis and may only be treated by existing agents having considerable side-effects, such as β-interferon. The compounds of the present invention allow a more targeted stimulation of APP and consequent removal of debris which may be highly valuable in such cases. Cell debris is also believed to contribute to Alzheimer's, Parkinson's and Huntinton's diseases.

The tonic effect of the compounds of the present invention in older subjects may also be explicable as a result of a cleanup mechanism. As subjects age, a greater proportion of cells suffer programmed cell death due to telomere reduction and apoptosis. At the same time, the level of clean up mechanisms such as APPs and is the effectiveness of the immune system typically declines. This may lead to a build up of debris and a susceptibility of infection, these factors then leading to degenerative diseases and conditions such as heart attacks. By prophylactic treatment with the compounds of the present invention, the immune system and APP levels may be stimulated reducing the debris buildup and causing the immune system to rid the body of infections before catastrophic events such as bursting of blood vessels causes conditions such as heart attacks.

In a similar way to that seen in aging subjects, those suffering from chronic disease may experience a build up of biological debris from both host cells and infectious agents. The compounds of the present invention may be administered to speed recovery and improve quality of life in such cases. This mechanism is also suitable for speeding the recovery of any subject after events such as surgery, burns or sepsis.

The immune stimulation and cleanup effect of the compounds of the invention may be used in combination with other drugs, particularly to improve the quality of life of subjects having compromised immune function resulting from a primary condition or the treatment therefore. For example, patients suffering from HIV and related conditions may be treated with one or more antireteroviral agents in order to treat or control the disease. Examples of these include reverse transcriptase inhibitors and protease inhibitors such as zidovudine, didanovine, zalcitabine, stavudine, lamivudine, nevirapine, delavirdine, indinavir, ritonavir, nelfinavir, hydroxyurea kolchicine, AZT and 2′, 3′-dideoxyinosine (ddI). In combination with this treatment, the compounds, compositions or medicaments of, the invention may be administered in order to purge and stimulate the patients remaining immune function.

Similarly, subjects suffering from a hyperplastic or neoplastic disease such as cancer or leukemia may be treated with one or more cytotoxic agents (such as nucleoside analogues), by surgery, external beam irradiation and/or radionuclide therapy. In such cases, the immune system of the subject is generally suppressed as a side effect of the therapy. The immune system may, however, be boosted by administration of the compounds of the present invention in order to provide the subject with lower susceptibility to infection during and after the primary therapy. In addition, the compounds or compositions of the present invention may be administered to stimulate or focus an immune response (particularly, for example by teh stimulation of macrophages) against any remaining tumour cells, micro-tumours or micro-metastases in order to provide more complete remission of the disease. Such treatment may be carried out during or after treatement by other agents or interventions.

The compounds of the present invention may also be used to stimulate the destruction (particularly by macrophages/monocytes) of micro-tumours and thereby prevent the formation or spread of neoplastic disease. This will apply particularly in older subjects (see below) or those considered as having a predisposition to neoplastic disease (e.g. due to hereditiy; exposure to predisposing chemical or physical environments, such as carcinogens, ionising radiation, etc; previous treatment for neoplastic disease; results of genetic testing etc).

In a further preferred aspect, the present invention therefore provides a method for the treatment of a mammalian (preferably human) subject comprising administration of a compound of formula I or a salt thereof as defined herein, in combination with another drug and/or treatment regime. Preferably, the method is a method for the treatment of a viral, hyperplastic or neoplastic disease, more preferably for the treatment of HIV, cancer or leukaemia. The other drug is preferably an antiviral, such as those listed herein or an antineoplastic agent-such as a radiopharmaceutical or chemotherapeutic (e.g. asparaginase, bleomycin, cisplatin, cladribine, cyclophosphomide, cytrabine, dacarbazine, daunorubicin, doxorubicin, etoposide, fluorouracil, hydroxyurea, mercaptopurine, mustine, methotrexate, procarbazine, or vinblastine). The other treatment regime is preferably surgery and/or external beam irradiation. In this method, the compound of the present invention will typically be formulated as a pharmaceutical, either as the sole active agent or in combination with at least one other drug agent and will be administered prior to or preferably consecutively with or after the other drug or treatment.

In a preferred embodiment, the invention also provides a method of prophylaxis against the development of cancer or other neoplastic disease comprising administration of a compound of the invention. Where symptoms such as fatigue or lethargy are the result of old age or viral, bacterial or fungal infection or the symptoms or treatment of hyperplastic disease such as cancer, the compounds of the present invention may be administered either as a pharmaceutical, or as an additive in, for example a “functional food”. Where the cause is a medical condition or treatment, the compound of the invention will generally be taken in the form of a pharmaceutical. Where, however, the cause is simply the result of the general build up of unwanted debris in old age, the compounds of the present invention will preferably be taken in the form of a functional food or dietary supplement for convenience and ease of compliance.

In a preferred aspect, the present invention therefore provides a method of tonic treatment of an aging mammalian (preferably human) subject, or a subject suffering from the aftereffects of infection, disease or treatment, comprising administration of a compound of formula I or a salt thereof as defined herein. Where the subject is an aging human, they will preferably be at least 60 years of age, more preferably at least 70 and most preferably at least 75. The subject may be suffering from an identifiable viral, immune-deficient, autoimmune or allergic disease or condition, or may be a generally healthy subject in these or all respects wishing for a boost in physical or mental energy or in immune function or a reduction in fatigue or lethargy. The invention also provides for the use of the compounds of the invention in the manufacture of a tonic medicament suitable for use in such methods.

The present invention will now be illustrated by the following, non-limiting examples and the attached Figures in which;

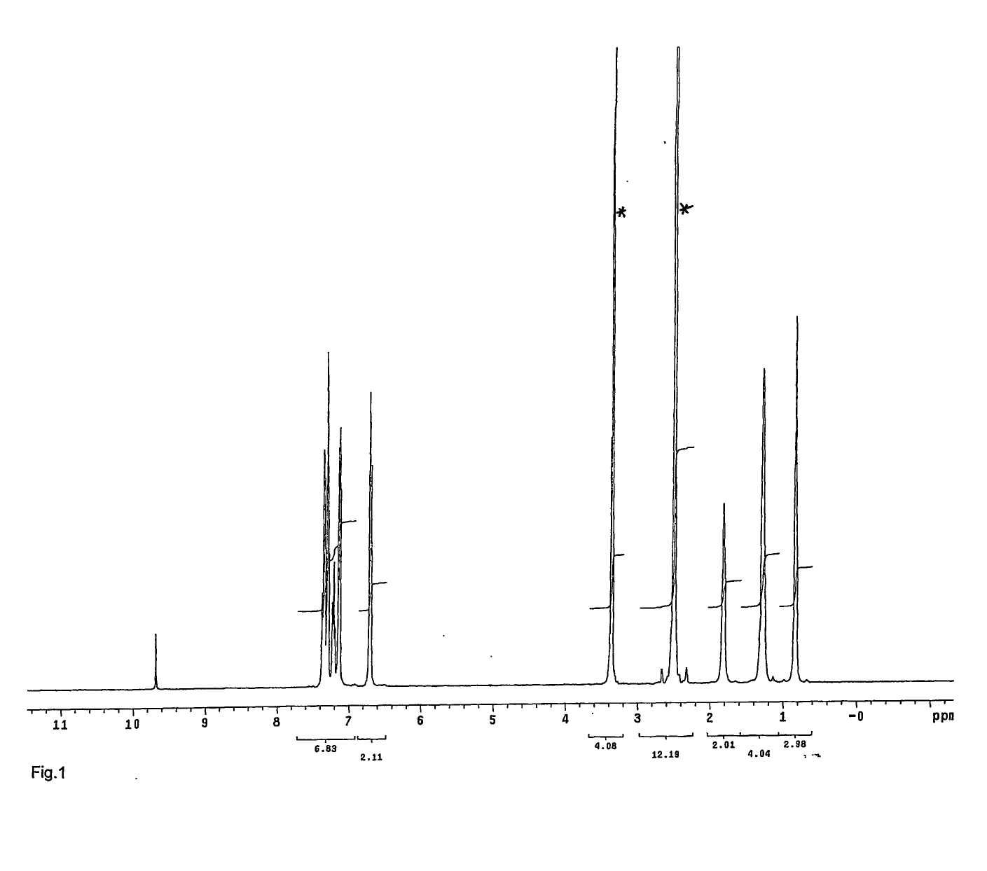

FIG. 1 shows the full proton NMR spectrum of 4-OH—OPB;

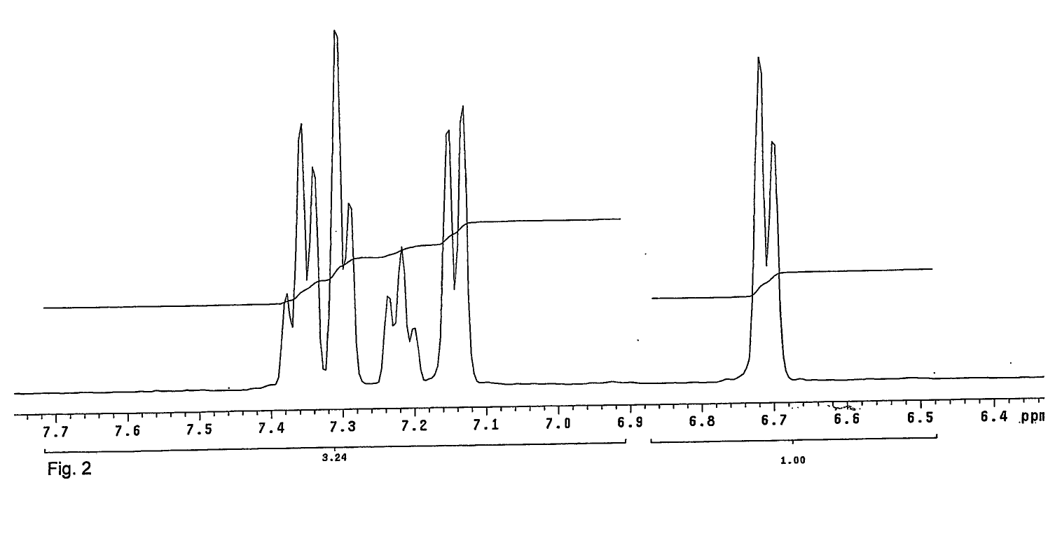

FIG. 2 shows an expansion of the aromatic region of the proton NMR spectrum of 4-OH—OPB;

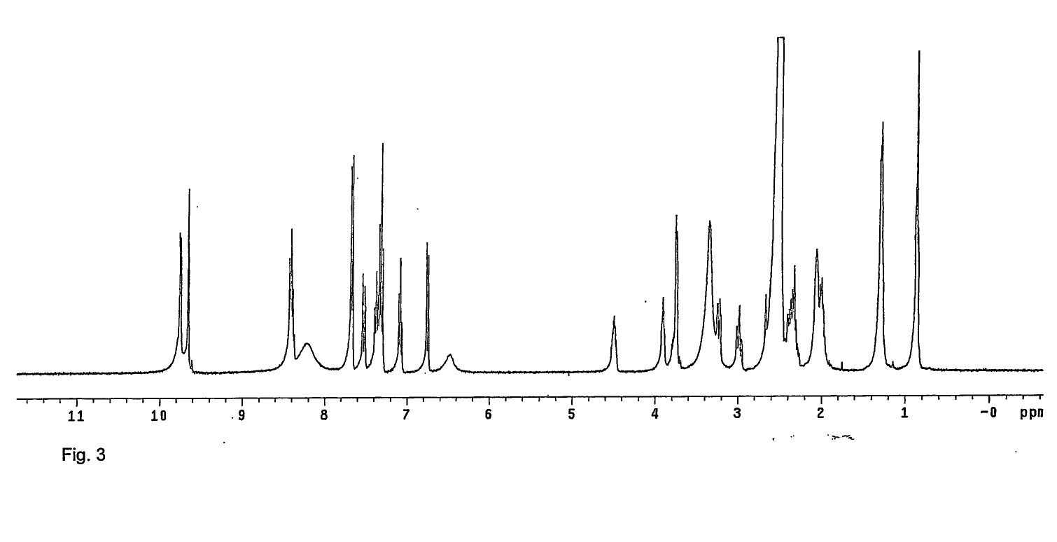

FIG. 3 shows the 1GSH derivative of 4-OH—OPB (4-OH—OPB-lGSH);

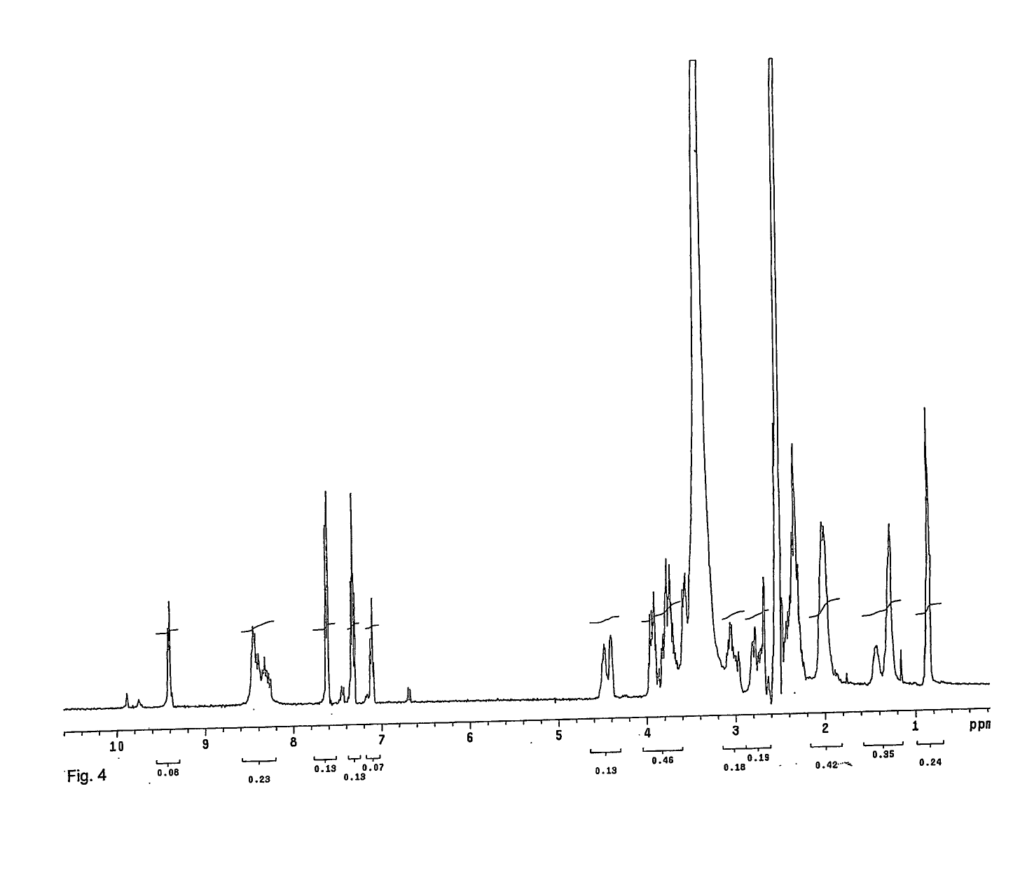

FIG. 4 shows the 2GSH derivative of 4-OH—OPB (4-OH—OPB-2GSH);

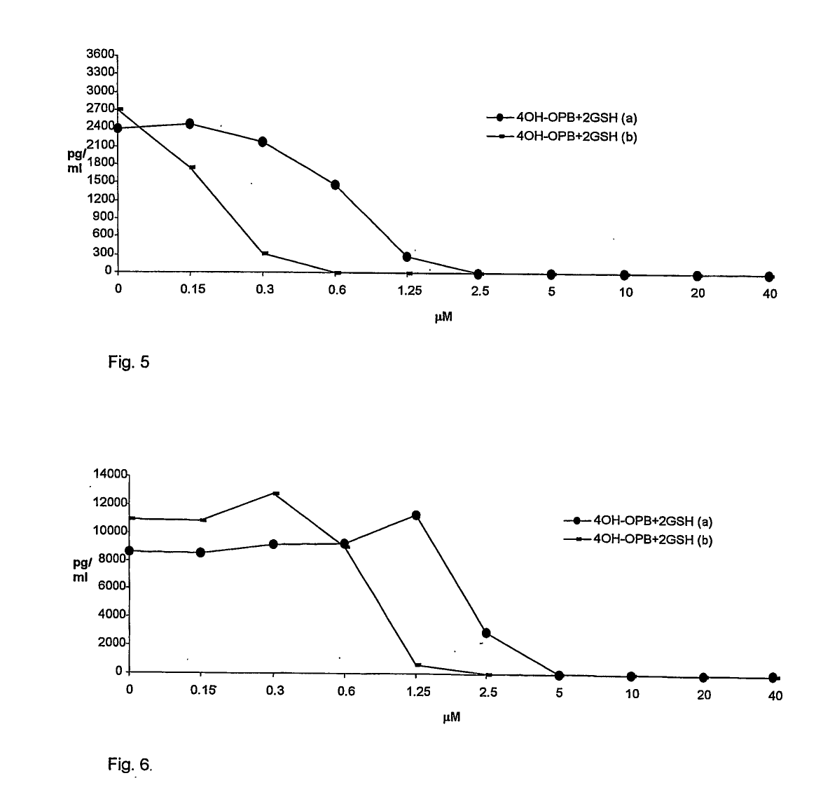

FIG. 5 shows the effect on Interleukin 6 (IL-6) production of mononuclear cells (MNC) after incubation with 4-OH—OPB-2GSH;

FIG. 6 shows the effect on Granulocyte Colony-Stimulating Factor (GM-CSF) production of mononuclear cells (MNC) after incubation with 4-OH—OPB-2GSH.

### EXAMPLES

1H-NMR were recorded on a Bruker 300 MHz spectrometer with CDCl₃ as solvent. HPLC was performed with a Gynkotek pump equipped with a Symmetry C-18, 5 mm, 3.9×150 mm column and a Gynkotek UVD 170S detector set at 254 nm. Gradient: 1% TFA in water/acetonitrile 70/30 to 0/100 in 8 min.

### Example 1

### Synthesis of Starting Material—40H OPB

To a 1-litre round bottom flask with magnetic stirring is charged methanol (450 ml) and oxyphenbutazone hydrate (90.0 g, 0.26 mol). The solution is stirred at ambient temperature and sodium hydroxide solution (2M, 13.5 ml) is added. Hydrogen peroxide (30%, 180 ml) is added drop wise over 10 min. The resulting clear pale yellow solution was stirred for 24 h. The resulting suspension was cooled on an ice bath for 2.5 h and the mixture filtered through a glass filter and sucked dry. The light brown crystals were washed carefully on the filter with MeOH/water (1:2, 200 ml), sucked dry and washed once more with 100 ml of the same solvent mixture. The product was allowed to dry on the filter over night. The crude product was then transferred to a 200 ml round bottom flask, diethyl ether (200 ml) added and the resulting suspension stirred vigorously for approximately 5 min. The mixture was filtered and sucked dry on the filter. The appearance of the product was pale pink after the ether treatment. Crude yield 53 g. The ether treatment procedure was repeated once more with 150 ml of ether. The now almost white material was dissolved in methanol (330 ml) to give a red solution. Water (350 ml) was charged slowly over 35 min to give a white suspension. The solid was collected on a glass filter and dried in vaccuo at 30° C. over night to give 4-OH OPB as a pale pink solid, 31 g, 35 w. HPLC>98%. ¹H NMR. (see FIG. 1) confirms identity with reference sample.

### Example 2

### Thiol Derivitisation

The 4OH-OPB of Exmample 1 was derivatised with glutathione by incubation in glutathione (GSH) solution followed by purification. Conditions were chosen such that approximately equaly quantities of the mono-glutathione substituted product (4OH-OPB-1GSH) and di-glutathione substituted product (4OH-OPB-2GSH) resulted.

Incubation

4OH-OPB (170 mg) was dissolved in PBS (100 ml, formulated as below) additionally containing 1.5 mM glutathione.

The solution was incubated for 30 minutes at 37° C. and the reaction followed by Mass Spectrometirc analysis.

Purification

An analytical HPLC run (C18 reversed phase column) was performed to validate the products formed (determination by Mass Spectrometry). The 40H-OPB-lGSH and 40H-OPB-2GSH were then purified by loading all 100 mL of the incubation mixture on a preparative column (C18, reversed phase column). Both analytical and preparative runs were eluted with gradient eluents, running from 0% acetonitrile to 67% acetonitrile (in deionised water) in the presence of 0.1% TFA to keep the pH at 2. During the preparative run, fractions were collected (peaks) and checked for the right product by Mass Spectrometry. Finally, the identified products were dried under vacuum leaving products with ˜99% purity (as judged by MS).

PBS=(phosphate buffered saline, pH 7.5) NaCl, 8.2 g; Na₂HPO₄.2H₂O, 1.9 g; NaH₂PO₄.H₂O, 0.3 g; Na⁺, 163, 9 mM; Cl⁻, 140, 3 mM; HPO₄²⁻, 10, 9 mM; H₂PO₄⁻, 1, 8 mM, Braun Melsungen AG.

The NMR spectra of the products of the above reaction are shown in FIGS. 3 and 4. FIG. 3 is the spectrum of a product with molecular weight corresponding to addition of 1 GSH, FIG. 4 is the specturm of a product with molecular weight corresponding to the addition of 2 GSH moieties.

### Example 3

### Suppression of Cytokine Production

Two batches of the 4OH-OPB-2GSH, as prepared in Example 2, were incubated with isolated human mononuclear cells (MNC) derived from peripheral blood from healthy volunteers. Production of the cytokines Interleukin-6 (IL6) and Granulocyte Colony-Stimulating Factor (GM-CSF) was measured. The results (shown in FIGS. 2 and 3) indicate that 0.5-5 μM of product is sufficient to completely block production of the measured cytokines.

1. An optionally substituted compound of the formula I or a salt thereof; 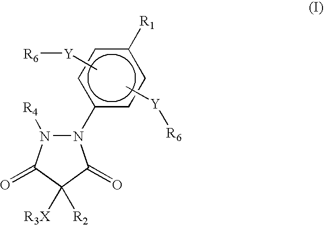
wherein R₁ is O or S, R₂ is hydrogen or more preferably an C₁-C₁₀ organic group attached by a carbon atom, X is H, O, OO, S or SS R₃ is absent where X=H, is hydrogen or is a hydroxyl or thiol protecting group, R₄ is a hetero- or preferably homo-cyclic aryl group, optionally substituted with a further group R₅ one Y group is S and the other is either H (in which case only one R₆ group is present) or S; and R₆ is, independently, an organic group of molecular weight up to around 500 amu.

2. A compound as claimed in claim 1 wherein R₂ is an optionally substituted alkyl, alkenyl, alkynyl, alkaryl, aralkyl, arylsulphonylalkyl or aralkenyl group, R₃ is hydrogen or a C₂-C₇, acyl, or alkaryl group, R₅ is an alkyl, alkenyl, alkynyl, OH, O-alkyl, thio, thioalkyl, halo, or primary, secondary, tertiary or quaternary amino group and R₆ is a substituted or unsubstituted alkyl, alkenyl, alkynyl, alkaryl, aralkyl, alkyl ester, alkyl amide, alkyl acid, polyol, sugar, oligo(alkylamide), oligo(alkylester), or oligopeptide group.

3. A compound as claimed in claim 1 of formula III; 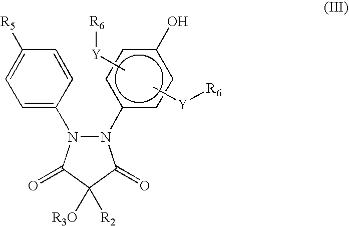
wherein Y, R₂ and R₃ are as described in claim 1 or claim 2 and R₅ is hydrogen or an alkyl, alkenyl group, OH, O-alkyl, O-acyl, SH, S-alkyl, S-acyl, halo, or amino group.

4. A compound as claimed in claim 1 of formula IV, or a salt thereof; 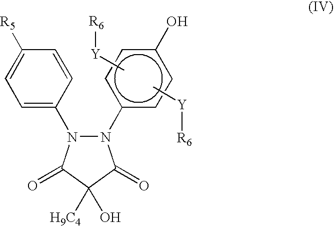
wherein R₅ is hydrogen or OH and each R₆ is, independently, a cysteine or a glutathione moiety attached via the sulphur atom thereof.

5. A compound as claimed in claim 1 wherein both Y—R₆ groups are S—R₆.

6. A compound of claim 1 wherein the two Y groups are ortho to group R₁.

7. A compound of claim 1 wherein one Y group is ortho to gtoup R₁ and one Y group is meta to gtoup R₁.

8. A compound formed by reaction of a thiol (R₆—SH) with a starting material of formula II 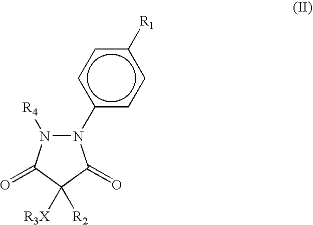
Wherein R₁, R₂, R₃, R₄, R₆ and X are as defined in claim 1, or protected derivatives or precursors thereof.

9. A method for the synthesis of a compound of claim 1, said method comprising reaction of a thiol (R₆—SH) with a starting material of formula II 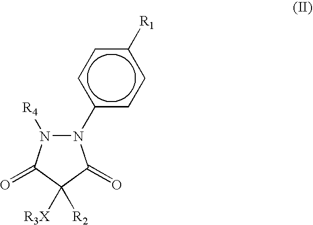
Wherein R₁, R₂, R₃, R₄, R₆ and X are as defined in claim 1, or protected derivatives or precursors thereof.

10. A method of treatment of a mammalian subject comprising administering a compound as claimed in claim 1.

11. A method for the for treatment, prevention or control of a viral, autoimmune, neoplastic, inflammatory and/or allergic condition, said method comprising administration of a compound as claimed in claim 1.

12. A method of tonic treatment of an aging mammalian subject, or a subject suffering from the aftereffects of infection, disease or treatment, said method comprising administration of a compound as claimed in claim 1.

13. A compound as claimed in claim 1 for use in therapy.

14. The use of a compound as claimed in claim 1 in the manufacture of a medicament.

15. A pharmaceutical composition comprising a compound as claimed in claim 1 and at least one pharmaceutically acceptable excipient, carrier or diluent.

16. A functional or fortified food comprising a compound as claimed in claim 1 formulated in an edible food or potable beverage product.
